(19) United States
(12) Patent Application Publication
(10) Pub. No.: US 2004/0015082 A1
(43) Pub. Date: Jan. 22, 2004

(54) Ultrasonography system and acoustic probe therefor

(76) Inventor: Jean-Louis Vernet, Paris (FR)

Correspondence Address: LOWE HAUPTMAN GILMAN & BERNER, LLP, 1700 DIAGNOSTIC ROAD, SUITE 300, ALEXANDRIA, VA, 22314, US

(21) Appl. No.: 10/399,945

(22) PCT Filed: Oct. 19, 2001
(86) PCT No.: PCT/FR01/03251

(30) Foreign Application Priority Data
Oct. 24, 2000 (FR) 00/13632

(51) Int. Cl. A61B 8/02

(52) U.S. Cl. 600/450

(57) ABSTRACT

Echography system consisting of an echograph (4) with which is associated an acoustic probe (1) comprising at least one antenna composed of a plurality of acoustic transducers making it possible to sound-sweep a working volume in which the tissues to be examined are to be situated, or placed.

The system has a probe incorporating at least one synthetically processed antenna which is composed of one or more rigidly associated transducer alignments and which is displaced on a surface, ordinarily plane, during echography.

The invention relates to an echography system more particularly intended to be employed for medical imaging purposes. It also relates to an acoustic probe intended to equip such a system.

As is known, echography systems are commonly used to visualize the tissues constituting the organs of human beings, for examination purposes and more particularly for purposes of examinations performed in real time.

These systems employ acoustic probes fitted with one or more antennas which are composed of acoustic transducers making it possible to sound-sweep a delimited working volume inside which the tissues to be echographed are to be situated, or placed. The antennas are used to transmit ultrasound exploration signals and/or to recover the signals which are reflected by the tissues illuminated by the exploration signals. The current trend is to produce probes whose antennas comprise a large number of transducers, with narrow emission beam, so as to be able to obtain accurate images from targets of relatively large dimensions. However, the dimensioning and the cost of linking transducers of a probe to the echograph, which serves this probe, become unacceptable when the intended number of transducers is very large. Now, it is possible to produce probes which have, for example, 2 500 transducers arranged according to a 50 by 50 rectangular matrix.

The invention therefore proposes an echography system making it possible to obtain high image accuracy, with a probe whose antenna acoustic transducers are served by a markedly lower number of linking wires than that of a system as envisaged hereinabove. This invention allows the installation of a link, between an associated probe and echograph, which exhibits high performance and which is technically acceptable, insofar as it may be produced in a fairly compact form and at a reasonable cost.

The acoustic probe comprises at least one antenna composed of a plurality of acoustic transducers making it possible to sound-sweep a working volume in which the tissues to be examined are to be situated, or placed. The echograph is programmed in such a way as to process the acoustic signals reflected by the tissues which are picked up by the transducers, after at least some of these transducers have previously emitted them, in such a way as to group these signals picked up into channels and to extract therefrom echographic images which can be employed by a user.

According to a main characteristic of the invention, the system is equipped with an acoustic probe comprising at least one antenna, composed of one or more rigidly associated transducer alignments making it possible to sound-sweep a working volume in which tissues to be examined are situated, or placed. This antenna is intended to be displaced, along at least one dimension of a surface, during echography and within the context of an antenna processing, of synthetic type.

According to an embodiment of the invention, the probe comprises at least one antenna, composed of one or more rigidly associated transducer alignments, which is mechanically displaced in a plane or along a curved surface, during echography.

According to a variant embodiment of the invention, the probe comprises at least one antenna, composed of one or more rigidly associated transducer alignments, which is intended to be displaced manually in a plane or along a curved surface, during echography.

According to a particular embodiment of the invention, the probe comprises at least one antenna intended to slide on a membrane functioning as acoustic interface with regard to the tissues to be echographed.

According to an embodiment of the invention, the indications relating to the positioning of the probe antenna or antennas are obtained by way of the drive mechanism.

According to a variant embodiment, the indications relating to the positioning of the probe antenna or antennas are obtained by way of at least one device for measurement which is independent of the means of displacement of probe antenna(s).

According to another variant embodiment, the indications relating to the positioning of the probe antenna or antennas are obtained by calculation in the echograph at the time of the programmed processing performed by the latter from the acoustic signals, reflected by the tissues, which have been picked up by the transducers during echography.

According to a particular embodiment, the transducers of a probe are distributed over three antennas each consisting of at least one transducer alignment and arranged in an H, the central antenna being employed for transmission and for reception so as to transmit and pick up the acoustic signals from which the echographic images are extracted, the lateral antennas being employed for reception so as to pick up the signals from which the indications relating to the positioning of the H assembly formed by these three antennas are extracted.

The invention also proposes an acoustic probe, for echography system, as defined in the main characteristic mentioned hereinabove.

According to another characteristic of the invention, the probe comprises a synthetic antenna composed of a plurality of acoustic transducers aligned in one or more columns on a surface and a system of addressing multiplexers making it possible jointly to select a portion of the transducers, on transmission or/and on reception, in such a way as to effect an apparent displacement of this portion by selective addressing of the multiplexers.

The invention, its characteristics and its advantages are specified in the description which follows in conjunction with the figures mentioned hereinbelow.

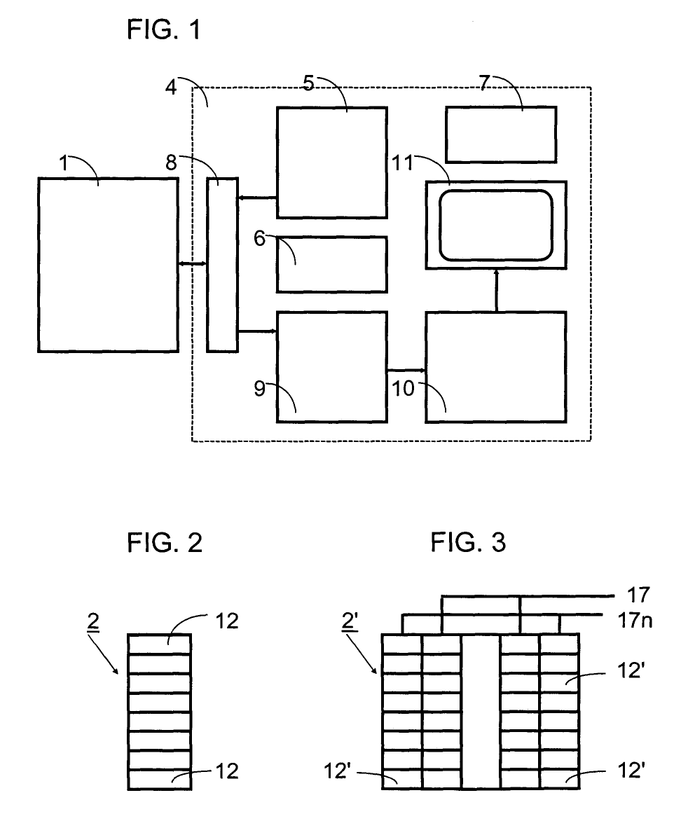

FIG. 1 depicts a known schematic diagram of an echography system.

FIG. 2 depicts a diagram relating to a first type of known probe antenna, with an alignment of transducers.

FIG. 3 depicts a diagram relating to a second type of known probe antenna, comprising several alignments of transducers.

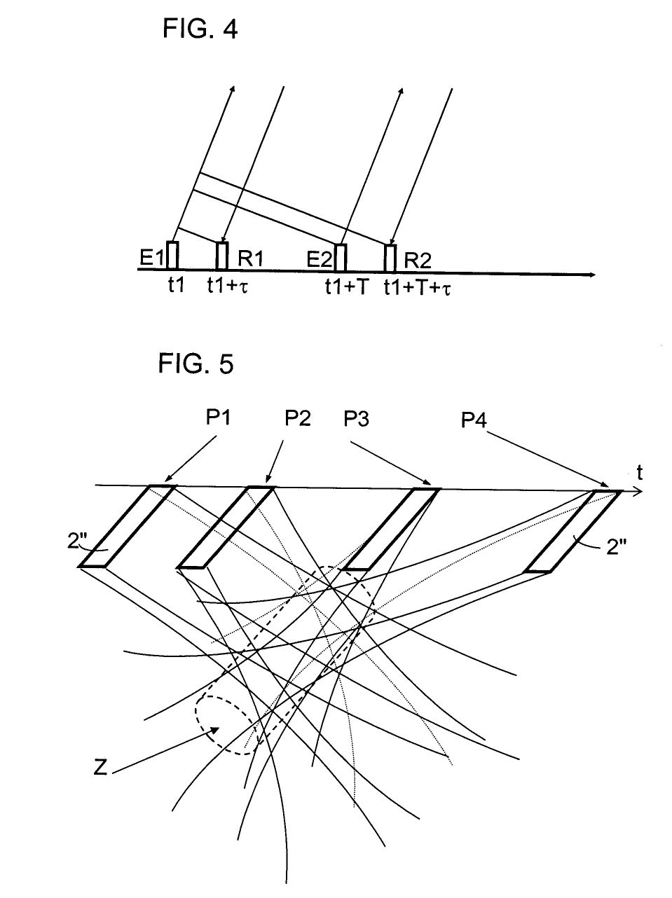

FIG. 4 depicts a defining schematic relating to a linear displacement of an antenna.

FIG. 5 depicts a basic diagram illustrating the scanning which can be effected with a probe antenna, according to the invention, displaced linearly within the context of an operation for obtaining an echographic image.

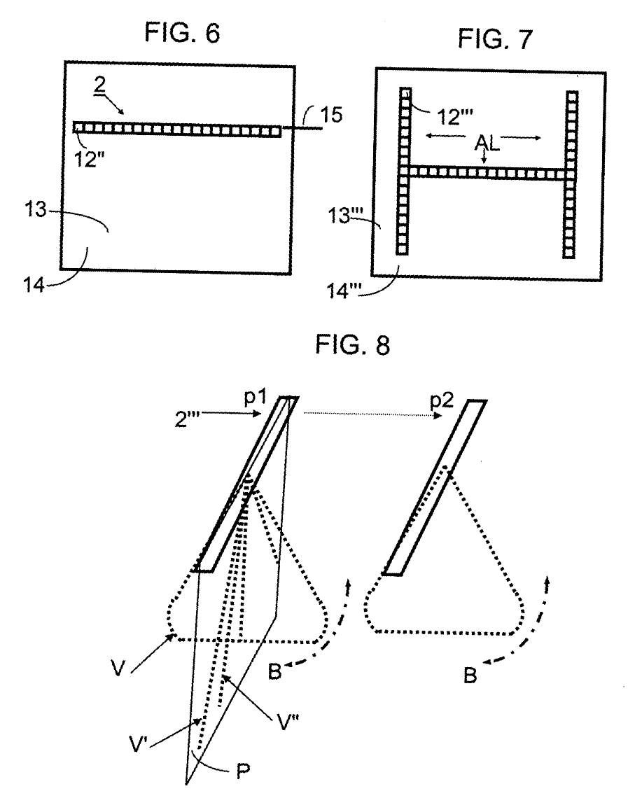

FIG. 6 depicts a basic diagram relating to a first embodiment of a probe layout for echography system, according to the invention.

FIG. 7 depicts a basic diagram relating to a second embodiment of a probe layout for echography system, according to the invention.

FIG. 8 depicts a basic diagram illustrating the modes of transmission for one of the transducers of a linear antenna.

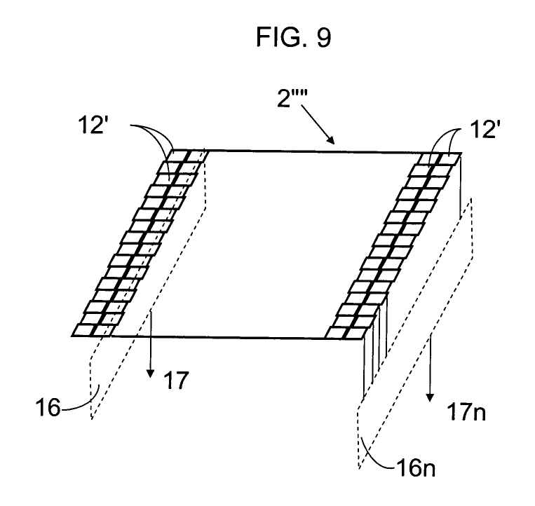

FIG. 9 depicts a diagram relating to an embodiment of a probe with multilinear antenna.

The echography system, shown diagrammatically in FIG. 1, conventionally comprises an echographic probe 1 including a plurality of transducers which define an antenna and which are intended to allow the sound-sweeping of a delimited working volume, in which the tissues to be echographed are to be situated, or placed. The probe 1 is organized, in a manner developed hereinbelow, so as to make it possible to explore targets which are situated at the level of the tissues and which are illuminated in a manner determined by the transducers. The latter are used both to transmit acoustic signals, to the targets, within the ultrasound region and also to recover these signals, after reflection by the targets.

For this purpose, the probe 1 is linked to an echograph 4 which comprises an emitter stage 5, wherein are produced the excitation signals which are sent to the transducers of the probe. This sending is performed according to a determined sequencing and with a determined periodicity, under the pulsing of a clock circuit 6, linked to this emitter stage in a conventional manner, not represented here. Control means, for example of keyboard or desk type, of a man/machine interface 7 allow a user to act, on the basis of his requirements, on various constituent elements of the echograph and possibly on the probe.

In the emission phase, excitation signals are transmitted, in the form of pulse trains, to the transducers of at least one antenna of the probe, from the emitter stage 5 and by way of a separator stage to which a receiver stage 9 is also linked. These excitation signals are transformed into ultrasound pulse signals at the level of the transducers of the probe antenna or antennas. The separator stage 8 makes it possible to prevent the excitation signals from blinding the receiver stage 9. The reflected ultrasound signals which are picked up by the transducers in the reception phase are taken into account by the receiver stage where they are organized in such a way as to be grouped together in reception channels. The grouping is carried out in a manner determined as a function of choices made available to the user, in particular for focusing purposes. A signal processing stage 10 makes it possible to translate the signals provided by the receiver stage into signals that can be employed by the user, for example into echographic images that may be presented on a display screen 11. As is known, the operation of the echograph is controlled by way of a programmed management unit which may possibly be merged to a greater or lesser extent with the processing stage 10. This operation is temporally governed by the clock circuit 6, in conjunction with the programmed management unit.

FIG. 2 depicts an acoustic probe, of 1D type, which comprises an antenna 2 composed of an alignment of ultrasonic transducers 12. The number of aligned transducers may be large and an alignment comprises, for example, 128 transducers.

According to the invention, provision is made to displace such a 1D probe antenna at a determined speed over a surface, ordinarily plane or curved, as required. The displacement of the probe antenna is used to sound-sweep a working volume by utilizing the possibilities of selective activation of the aligned transducers 12. An image whose definition is comparable in both dimensions can be obtained, since the transducers may be regarded as if they were dispersed over a surface and they may therefore be utilized synthetically, by utilizing the motion of the antenna, both on transmission and on reception.

A signal, emitted at an instant t1 and at a position E1 along the path followed by the antenna of the probe, is received at the time t1+τ and at a position R1, as illustrated in FIG. 4.

A signal, emitted at an instant t1+T and at a position E2, is received at the time t1+T+τ′ and at a position R2, τ′ being different from τ. The variation in delay time τ′-τ induces a phase variation, between the receptions at the positions R1 and R2, at a given frequency. This phase variation is twice the phase difference between two sensors of a fixed reception antenna of length E2-E1. The recording of the signals picked up, obtained from identical signals, makes it possible to perform a processing for rephasing the signals originating from a determined source point, in a zone determined by the pattern of the transmission antenna.

In the case of the probe mentioned in conjunction with FIG. 2, whose antenna 2 is assumed to consist of 128 sensors half a wavelength apart and therefore having a total length corresponding to 64λ, the image obtained, if the probe antenna is displaced by 32λ, therefore corresponds to an image which would be provided by a plane probe antenna whose surface area would be equal to 64λ×64λ.

It is also possible to substitute a probe with antenna 2′ comprising several rows of transducers, such as is shown diagrammatically in FIG. 3, by the probe with linear antenna 2 envisaged hitherto. This antenna 2′ is composed of a plurality of alignments each comprising a certain number of transducers 12′, this number possibly being different from one row to another. The number of transducers per alignment corresponds for example to the envisaged number of transducers for the probe 2, each transducer being controlled individually by the programmed management unit of the system to which it is linked via a preferably shared wire link, such as 17 or 17n.

A rectilinear displacement of an antenna of this kind is shown diagrammatically in FIG. 5, where four successive positions P1 to P4 of the antenna, referenced 2″, are represented. An exemplary beam which can be obtained for each of these antenna positions is also illustrated, the various beams are here assumed to be oriented toward a central zone of interest Z.

It is possible to vary the pattern of the antenna on transmission so as to concentrate the beam onto a particular zone of the space to be examined, by driving the transducers with suitably delayed signals. It is also possible to vary the pattern of the antenna in reception so as to improve imaging, for example by creating zeros in determined directions.

The displacement of the antenna 2″ is intended to be carried out at determined speed and under conditions practically similar to those mentioned hereinabove, in conjunction with the antenna 2. It makes it possible easily to obtain images having comparable definitions, in a zone that the operator can choose in the tissues examined. It is vital to be able to accurately locate the antenna of a probe during displacement, whether this antenna be linear, such as 2, or multilinear, such as 2′. This locating may be only relative, it then involves accurately knowing the position of the probe antenna, at a given instant, during the displacement which it performs, with respect to its initial departure position, at the start of displacement. An accuracy of the order of 3/100 mm is for example to be envisaged, in the case where a frequency of the order of 5 MHz is used.

The knowledge of the positioning of the probe antenna may be obtained with the required accuracy, when this probe antenna is displaced by way of an accurate and faithful system, which may be a mechanical system and, for example, a system driven by one or more stepper motor(s). The indications relating to the positioning of the probe antenna or antennas may then be obtained by way of the drive mechanism.

It is also possible for the position of a probe antenna to be known accurately by means of one or more measurement devices, for example of accelerometer type, independent of the antenna displacement means, as also envisaged by the invention.

An exemplary probe with linear antenna, which can be displaced mechanically, is illustrated in FIG. 6. It is assumed therein that the drive mechanism makes it possible to know the position of the probe antenna to better than λ/8, i.e. practically 0.03 mm for an antenna of a probe operating with a wavelength of 0.3 mm. The probe is assumed to operate at 5 MHz and to comprise an alignment whose length is 19.2 mm, this probe antenna being composed of 128 transducers 12″, which are λ/2 apart and whose width is practically equal to λ. The probe antenna can displace parallel to itself under the action of the mechanism, not represented, by gliding over a relatively rigid membrane 13 stretched over a frame 14. The displacement of the probe antenna is, for example, limited to a length of the order of a centimeter. The membrane acts as an acoustic interface in relation to the tissues to be examined, which here are assumed to be under it. The transducers of the probe are able to be linked by a flexible link 15 to the echograph proper. The linear antenna has a maximum directivity approximately equal to 0.9° or 1/64 radian in a plane, this therefore corresponding to a resolution of 3 mm at a distance of 20 cm and a resolution of 0.625 mm at 4 cm. It has a directivity in the other plane of 30°, resulting in a sound-swept zone being obtained in the other plane, which is 2 cm at a range of 4 cm and 10 cm at a range of 2 cm.

The directivity which can be obtained may be appreciably greater in the case of an antenna consisting of several rows of transducers, it being possible for this antenna to be used spatially in various ways, as set forth earlier. It may moreover be used in such a way as to increase the signal-to-noise ratio.

The displacement of the above-envisaged linear antenna, over a distance of 1 cm, is equivalent to the obtaining of a synthetic antenna whose directivity is 1/64 radian in both directions, this therefore corresponding to an antenna gain of 4 096 and a resolution cell size of 0.6×0.6, i.e. 0.36 mm² at 4 cm.

It is also possible to envisage making a probe with synthetically processed antenna which consists of a linear or multilinear antenna whose spatial position is obtained by employing a measurement device which is independent of the antenna displacement drive system and which is not one of the conventional measurement devices, envisaged earlier. This is envisaged in particular in the case where the displacement of the probe antenna of an echography system is effected manually by the user.

An exemplary probe intended for such a case is shown diagrammatically in FIG. 7, the antenna of this probe is composed of three linear antennas AL arranged in an H and each comprising one and the same number of transducers 12″′. Each linear antenna AL is, for example, constructed in the same manner as the linear antenna described in conjunction with FIGS. 2 and 5. It is assumed to exhibit the same characteristics. It is also assumed that the H assembly, formed by the three linear antennas AL of the probe, can displace over a short distance by gliding over a membrane 13″′ which is carried by a frame 14″′ and under which membrane the tissues to be examined are situated. As previously, the transducers of the probe can be linked by a flexible link to the echograph proper. The alignment of transducers constituting the central antenna AL, situated between the two lateral antennas, is used for visualization in the same manner as the alignment of transducers 12″ of the antenna 2. The two lateral antennas AL are used for measurement of the motion of the probe antenna. This measurement implies that the echoes to be located at the level of the tissues under examination are fixed or may be regarded as fixed, when the probe antenna is displaced, in such a way that the coherence of the reception signals can be ensured and that consequently good image resolution and good accuracy of the positioning measurements may be obtained.

In the case of a linear antenna 2″′, corresponding for example to the central element AL mentioned hereinabove, it is possible to obtain the formation of channels V, of specific shape, such as are depicted for a given transducer in a first antenna position p1 of two successive positions p1, p2 shown in FIG. 8. These channels V, V′, V″ exhibit great slenderness in the plane P, along the alignment of transducers of the antenna 2″′ and perpendicularly to the plane defined by these transducers. They also exhibit a significant width in the other direction, as shown for one of the channels in each of the two positions p1 and p2 assumed to be reached successively by translating the antenna.

If it is assumed that around one hundred channels are formed with the aid of a central antenna AL which is used for visualization, and that the emission recurrence period is around 250 μs, for a range of 20 cm, it is then necessary for the speed of displacement of the assembly consisting of the three linear antennas AL not to exceed 100×250=25 ms for 0.15 mm. This therefore corresponds to a speed of 6 mm/s and to a scan carried out in slightly more than three seconds, if the displacement is performed over 2 cm. It is then necessary for the elements of the tissues, which serve in the formation of the echoes, not to have moved over a distance, corresponding to the side of the distance bin, by more than 0.6 mm, if the range taken into account is 4 cm, as envisaged hereinabove.

The constraints set forth hereinabove are also to be taken into account with the linear antenna envisaged earlier, regardless of the manner, manual or mechanical, in which this linear antenna is displaced.

When the displacement of a probe antenna, of 1D or 1.5D type, is mechanical and is carried out in a sufficiently determined manner, so that the position of the probe antenna can be regarded as known perfectly at any instant during its displacement, the only operation which has to be undertaken, before processing, is to place the reflected signals in memory, in such a way as to be able subsequently to construct the synthetic channels.

When the displacement of the antenna is not performed under the conditions envisaged hereinabove, for example because it is carried out manually, it is nevertheless possible for the position of the probe antenna to be known accurately. Such is the case, if the latter forms part of an assembly of antennas, of the type having three linear antennas AL mounted as an H, which is described earlier. The measurement of the displacement of the assembly constituted by the three H-shaped antennas is carried out based on the two lateral antennas, used only for reception, and by way of correlation measurements performed on the sensors of these antennas, from recurrence to recurrence. The use of two lateral antennas situated at the ends of the central antenna makes it possible to increase the accuracy of the measurement of rotation of the assembly constituted by the three antennas.

Whatever may be the case, once the signals recovered are recorded in memory, it is possible to form channels throughout the space that the antenna has sound swept, during its displacement. The results obtained correspond to those which could be achieved with a 2D probe composed, as is known, of a large number of transducers dispersed according to a rectangular matrix of large dimensions. As indicated earlier, the processing of the signals recovered is performed by rephasing, the signals originating from a determined source point, in a zone determined by the pattern of the probe antenna, as carried out conventionally in the field of radars for ground imaging from a satellite or an airplane. The motion estimation is carried out by measuring the correlation between antenna sensors from one transmission to the other.

The invention also applies when the antenna is of the 2D type, as assumed for the antenna 2″″ of the probe depicted in FIG. 9 and that the scanning is carried out, not by mechanical means, but with the aid of multiplexers 16, . . . , 16n situated inside the probe. This makes it possible, in fact, to reduce the number of links 17, . . . , 17n between the transducers of the antenna and the remainder of the echography system.

Depending on the number and the type of necessary connections, it is possible to carry out a scan with a probe having an antenna, of 1D type, such as 2′, comprising a single row of transducers or with one of the antennas, of multilinear type, envisaged hereinabove, while benefiting from the advantage of a reduced number of linking cables between the probe and the system.

Additionally, in this configuration, it is possible to modify, by simply changing the addressing law for the multiplexers, the speed of displacement of the antenna portion used on transmission or on reception in an advantageous manner as regards the duration of formation of the image and the performance of the Doppler processing.

1. An echography system consisting of an echograph (4) with which is associated an acoustic probe (1) comprising at least one antenna composed of a plurality of acoustic transducers (12) making it possible to sound-sweep a working volume in which the tissues to be examined are to be situated, or placed, the echograph being programmed in such a way as to process the acoustic signals reflected by the tissues which are picked up by the transducers, after at least some of these transducers have previously emitted them, in such a way as to group the signals picked up into channels and to extract therefrom echographic images which can be employed by a user, said system being characterized in that it comprises a probe, of the type with synthetically processed antenna, with antenna (2 or 2′), composed of one or more rigidly associated transducer alignments, which is intended to be displaced, along at least one dimension of a surface, during echography.

2. The echography system as claimed in claim 1, in which the probe comprises at least one antenna, composed of one or more rigidly associated transducer alignments, which is mechanically displaced in a plane or along a curved surface, during echography.

3. The echography system as claimed in claim 1, in which the probe comprises at least one antenna, composed of one or more rigidly associated transducer alignments, which is intended to be displaced manually in a plane or along a curved surface, during echography.

4. The echography system as claimed in either of claims 2 and 3, in which the probe comprises at least one antenna intended to slide on a membrane (13) functioning as acoustic interface with regard to the tissues to be echographed.

5. The echography system as claimed in either of claims 2, 4, characterized in that it comprises a drive mechanism which provides the indications relating to the positioning of the probe antenna or antennas.

6. The echography system as claimed in one of claims 2, 3 or 4, characterized in that it comprises a device for measurements which is independent of the drive mechanism, in which indications relating to the positioning of the probe antenna or antennas are obtained by way of at least one device for measurement which is independent of the means of displacement of probe antenna(s).

7. The echography system as claimed in one of claims 1 to 4, characterized in that the echograph comprises means of calculation, in the echograph at the time of the programmed processing, of indications relating to the positioning of the probe antenna or antennas, from the acoustic signals, reflected by the tissues, which have been picked up by the transducers during echography.

8. The echography system as claimed in claim 7, in which the transducers of a probe are distributed over three antennas each consisting of at least one transducer alignment (AL) and arranged in an H, the central antenna being employed for transmission and for reception so as to transmit and pick up the acoustic signals from which the echographic images are extracted, the lateral antennas being employed for reception so as to pick up the signals from which the indications relating to the positioning of the H assembly formed by these three antennas are extracted.
